(19) United States
(12) Patent Application Publication
(10) Pub. No.: US 2010/0120149 A1
(43) Pub. Date: May 13, 2010

(54) CELL AGGREGATE-HYDROGEL-POLYMER SCAFFOLD COMPLEX FOR CARTILAGE REGENERATION, METHOD FOR THE PREPARATION THEREOF AND COMPOSITION COMPRISING THE SAME

(75) Inventors: Soo Hyun Kim, Seoul (KR); Young Mee Jung, Seoul (KR); Sang-Heon Kim, Seoul (KR)

Correspondence Address: OBLON, SPIVAK, MCCLELLAND MAIER & NEUSTADT, L.L.P., 1940 DUKE STREET, ALEXANDRIA, VA, 22314, US

(73) Assignee: KOREA INSTITUTE OF SCIENCE AND TECHNOLOGY, Seongbuk-gu (KR)

(21) Appl. No.: 12/614,561

(22) Filed: Nov. 9, 2009

(30) Foreign Application Priority Data
Nov. 7, 2008 (KR) 10-2008-0110395

(51) Int. Cl. C12N 5/00

(52) U.S. Cl. 435/396

(57) ABSTRACT

The present invention relates to a cell aggregate-hydrogel-polymer scaffold complex useful for cartilage regeneration which has a structure in which cell aggregates of differentiated chondrocytes are evenly dispersed in a hydrogel matrix, and the resulting hydrogel matrix is immobilized onto the surface of a polymer scaffold while filling up the pores thereof. Since the cell aggregate-hydrogel-polymer scaffold complex according to the present invention can efficiently induce the regeneration of cartilage tissue similar to natural cartilage and retain high mechanical strength, flexibility, and uniform morphology during the cartilage regeneration, it can be effectively used as a cartilage therapeutic agent for the repair of cartilage defects and injuries.

The present application claims priority from Korean Patent Application No. 10-2008-110395, filed Nov. 7, 2008, the subject matter of which is incorporated herein by reference in its entirety.

### FIELD OF THE INVENTION

The present invention relates to a cell aggregate-hydrogel-polymer scaffold complex useful for cartilage regeneration including cell aggregates of differentiated chondrocytes, a hydrogel matrix, and a porous polymer scaffold, where the cell aggregates are evenly dispersed in the hydrogel matrix, and the resulting hydrogel matrix is immobilized onto the surface of the polymer scaffold while filling up the pores thereof; a method for the preparation thereof; and compositions comprising the same for cartilage regeneration.

### BACKGROUND OF THE INVENTION

Arthritis is caused by chronic inflammation of the joint, accompanied by pain, swelling, and limited movement in the joints and connective tissue. It afflicts more than 80% of women older than 55 in the Republic of Korea. The most prevalent forms of arthritis are osteoarthritis and rheumatoid arthritis, both of which are progressive, degenerative diseases that lead to varying degrees of disability. The cartilage and bones of the joint undergo deterioration with the progress of the disease, followed by a loss of mobility and increased suffering caused by, among others, the rubbing of one bone against another.

The available therapies at present include palliative treatment, based on the use of analgesic or anti-inflammatory agents, and surgical therapy including partial or total joint replacement. Total joint replacement is routinely used for the knee, which is usually the most important joint afflicted by the disease. However, joint replacement is an expensive procedure that causes patient discomfort, serious potential post-operative morbidity, and other risks associated with surgery involving the opening up the joint. Joint replacement also has the drawback of limited durability, since the implanted prostheses only last for about 10-15 years.

Thus, research on new therapies for cartilage regeneration has been actively underway in the past few decades. Currently, various kinds of therapies including multiple drilling, microfracturing, abrasion, periosteal graft, and perichondral graft have been used for repairing cartilage defects and injuries, but their therapeutic effects were found to be very limited because they could only achieve regeneration of the fibrous cartilage. Further, cartilage autograft and allograft have also been used, but have disadvantages due to the limited donor-site or donor availability. Therefore, it is very important to regenerate damaged cartilage into a tissue that is histologically and biomechanically similar to natural cartilage for the prevention and treatment of cartilage defects.

Several studies have been conducted in an effort to overcome the above-mentioned limitations of the previously known therapies for cartilage regeneration. Thus, a method for the treatment of deep cartilage defects in the knee by autologous chondrocyte transplantation has been reported (Brittberg et al., N. Engl. J. Med. 331(14): 889-95, 1994). Since the above method has proved successful in obtaining regenerative cartilage tissue that is relatively similar to natural cartilage by culturing autologous chondrocytes, clinical trials using autologous chondrocyte transplantation have been steadily rising in the United States and Northern Europe. However, since the above method injects cultured chondrocytes in a suspension directly into a cartilage defect area, there have been problems in that the injected cells are easily washed out after the transplantation and, as a result, it is very difficult to maintain high cell density in the defect area. Further, cartilage matrix molecules generated from the transplanted chondrocytes exhibit fibrous cartilage-like characteristics different from natural cartilage, which is problematic in terms of the mechanical strength and long-term durability of the regenerated cartilage.

Furthermore, in case of producing artificial cartilage ex vivo in a certain shape and transplanting it into a cartilage defect area, there is a risk that the transplanted artificial cartilage may not completely adhere to the adjacent host cartilage in the defect area, leading to a reduction in mechanical strength. Therefore, in order to develop an effective treatment method for cartilage regeneration, the method should efficiently induce cartilage regeneration from the transplanted chondrocytes and, during the cartilage regeneration, the cartilage should retain high mechanical strength, flexibility, and uniform morphology. The present invention is directed to achieving the above objectives.

### SUMMARY OF THE INVENTION

One of the objectives of the present invention is to provide a cartilage therapeutic agent capable of inducing the effective regeneration of cartilage tissue that is similar to natural cartilage, while retaining high mechanical strength, flexibility, and uniform morphology, and a method of repairing cartilage defects and injuries by using the same.

One embodiment of the present invention relates to a cell aggregate-hydrogel-polymer scaffold complex useful for cartilage regeneration.

Another embodiment of the present invention relates to a method of preparing such cell aggregate-hydrogel-polymer scaffold complex.

Another embodiment of the present invention relates to a composition for cartilage regeneration which includes the cell aggregate-hydrogel-polymer scaffold complex as an effective ingredient.

### BRIEF DESCRIPTION OF THE DRAWINGS

The embodiments of the present invention will be described in detail with reference to the following drawings.

FIG. 1 is a schematic diagram illustrating the structure of a cell aggregate-hydrogel-polymer scaffold complex according to the present invention.

FIG. 2A is an optical microscope photograph of cell aggregates formed by hanging drop culture according to the present invention.

FIG. 2B is a fluorescent microscope photograph of cell aggregates formed by hanging drop culture, followed by continuous fluorescence depletion anisotropy (CFDA) labeling, according to the present invention.

FIG. 2C is an electron microscope photograph of cell aggregates formed by hanging drop culture according to the present invention.

FIG. 3A is an electron microscope photograph of a cell aggregate-hydrogel-polymer scaffold complex prepared by hanging drop culture according to the present invention.

FIG. 3B is an electron microscope photograph at high magnification (×1000) of a cell aggregate-hydrogel-polymer scaffold complex prepared by hanging drop culture according to the present invention.

FIG. 3C is a fluorescent microscope photograph of a cell aggregate-hydrogel-polymer scaffold complex prepared by hanging drop culture, followed by CFDA′ labeling, according to the present invention.

FIG. 3D is an optical microscope photograph of a cell aggregate-hydrogel-polymer scaffold complex prepared by hanging drop culture according to the present invention, which was removed from the cartilage defect area of a nude mouse 8 weeks after subcutaneous transplantation.

FIG. 4A is a fluorescent microscope photograph of cell aggregates formed by rotational culture, followed by CFDA labeling, according to the present invention.

FIG. 4B is a fluorescent microscope photograph of a cell aggregate-hydrogel-polymer scaffold complex prepared by rotational culture, followed by CFDA labeling, according to the present invention.

FIG. 4C is an optical microscope photograph of a cell aggregate-hydrogel-polymer scaffold complex prepared by rotational culture according to the present invention, which was removed from the cartilage defect area of a nude mouse 8 weeks after the subcutaneous transplantation.

### DETAILED DESCRIPTION OF THE INVENTION

The present invention provides a cell aggregate-hydrogel-polymer scaffold complex useful for cartilage regeneration.

The cell aggregate-hydrogel-polymer scaffold complex according to the present invention includes cell aggregates of differentiated chondrocytes, a hydrogel matrix, and a porous polymer scaffold, and has a structure in which the cell aggregates are evenly dispersed in the hydrogel matrix and the resulting hydrogel matrix is immobilized onto the surface of the polymer scaffold while filling up the pores thereof.

The cell aggregate-hydrogel-polymer scaffold complex of the present invention can be prepared according to a method including the following steps of:

1) differentiating cells having chondrogenic differentiation potential into chondrocytes while simultaneously clustering them to form cell aggregates of differentiated chondrocytes;

2) evenly dispersing the cell aggregates in a hydrogel matrix to prepare a cell aggregate-hydrogel complex; and

3) seeding the cell aggregate-hydrogel complex onto a porous polymer scaffold, to immobilize it onto the surface of the polymer scaffold while filling up the pores thereof.

Since the cell aggregate-hydrogel-polymer scaffold complex of the present invention utilizes fully differentiated chondrocytes in an aggregate form rather than free floating single cells, it is possible to efficiently induce chondrogenic differentiation through intracellular interaction between the aggregated cells. Further, in the cell aggregate-hydrogel-polymer scaffold complex of the present invention, the cell aggregates of differentiated chondrocytes are evenly dispersed in the hydrogel matrix, artificially creating a three-dimensional environment which is physiologically similar to natural cartilage, and thereby, improves the cartilage regeneration efficiency of the chondrocytes. In order to overcome the problem associated with the use of hydrogels, i.e., low mechanical strength of the hydrogel which makes it inappropriate for application to a large cartilage defect area, the cell aggregate-hydrogel-polymer scaffold complex of the present invention utilizes a biocompatible and biodegradable porous polymer scaffold in combination with hydrogels, and thereby, can retain high mechanical strength and flexibility with uniform morphology during cartilage regeneration. Therefore, the cell aggregate-hydrogel-polymer scaffold complex of the present invention can be effectively used as a cartilage therapeutic agent for the repair of cartilage detects and rapid regeneration of cartilage.

Hereinafter, the characteristics of the cell aggregate-hydrogel-polymer scaffold complex according to the present invention will be described in more detail.

The cell aggregate-hydrogel-polymer scaffold complex according to the present invention may be characterized as including cell aggregates of differentiated chondrocytes, a hydrogel matrix, and a porous polymer scaffold, and having a structure in which the cell aggregates are evenly dispersed in the hydrogel matrix and the resulting hydrogel matrix is immobilized onto the surface of the polymer scaffold while filling up the pores thereof.

Chondrocytes can maintain their differentiation potential and secrete chondrogenesis-related extracellular matrix (ECM) molecules in three-dimensional environments. When primary cultured chondrocytes or mesenchymal stem cells for chondrogenic differentiation are cultured in a two-dimensional culture dish, the cells lose the phenotypic characteristics of chondrocytes and are unable to maintain their differentiation potential. Therefore, in order to create three-dimensional culture environments essential for chondrogenic differentiation and chondrogenesis, the present invention utilizes fully differentiated chondrocytes in an aggregate form rather than single cells.

First, cells having chondrogenic differentiation potential are inoculated into a medium for chondrogenic differentiation and cultured for a certain time so as to differentiate them into chondrocytes. Suitable examples of cells having chondrogenic differentiation potential for the present invention may include mesenchymal stem cells and interstitial cells derived from bone marrow, muscle, adipose tissue, umbilical cord, amnion, or amniotic fluid; precursor cells derived from such cells that can be differentiated into chondrocytes; chondrocytes differentiated from such cells; primary chondrocytes isolated from cartilage tissue and the like, but are not limited thereto. The above cells can be used alone or as a mixture thereof. The isolation, proliferation, and differentiation into chondrocytes of the above cells can be carried out according to conventional methods well known in the art. Further, any medium for chondrogenic differentiation may be used for the present invention, so long as it is capable of differentiating the above cells into chondrocytes. In one embodiment of the present invention, DMEM (Dulbecco's modified Eagle's Medium) supplemented with 10% serum, 1% antibiotics, insulin, dexamethasone, ascorbic acid, and growth factors may be used as a medium for chondrogenic differentiation.

Upon chondrogenic differentiation, the differentiated chondrocytes are clustered together to form cell aggregates. For the formation of cell aggregates, various types of methods, such as hanging drop culture, pellet culture, micromass culture, and rotational culture may be used. Among the above-mentioned methods, while the hanging drop culture, pellet culture, and micromass culture can form cell aggregates of a uniform size, the rotational culture can form cell aggregates of varying size. In one embodiment of the present invention, cell aggregates having an average diameter of more than 100 μm may be formed by using differentiated chondrocytes of more than 1×10⁵ cells according to one of the above methods.

For a successful seeding of the cell aggregates onto a polymer scaffold, it is necessary to regulate the average diameter of cell aggregates depending on the pore size of the polymer scaffold used. Specifically, the average diameter of cell aggregates can be regulated by adjusting the number of cells used in forming the cell aggregate. In one embodiment of the present invention, the cell aggregates have an average diameter in the range of from 10 to 1,000 μm. Considering that conventional polymer scaffolds used in tissue engineering have a pore size in the range of from 10 to 1000 μm, the cell aggregates of the present invention may be formed to have an average diameter in the range of from 10 to 800 μm. The number of cells used in the formation of cell aggregates having such a size can be varied according to the type of cell used or the size of a single cell. For example, chondrocytes or bone marrow-derived mesenchymal stem cells are subjected to primary culture in the number of 1×10³ to 1×10⁷ cells, out of which 1×10³ to 1×10⁶ cells may be clustered together and form cell aggregates having an average diameter in the range of from 10 to 800 μm. If the number of cells used in the formation of cell aggregates is lower than the above range, the thus formed cell aggregates may have an average diameter not exceeding 10 μm, which would be invisible to the naked eye. Thus, there is a risk of a large amount of cell loss in the course of forming and recovering the cell aggregates. Further, since the efficiency of chondrogenic differentiation is in proportion to the size of cell aggregates, it is required that the cell aggregates have an average diameter larger than a certain size. On the other hand, if the average diameter of cell aggregates is excessively larger than the pore size of a polymer scaffold (e.g., the average diameter exceeding 800 μm), the cell aggregates are not successfully introduced into the pores of a polymer scaffold, thereby making it very difficult to induce chondrogenic differentiation inside the polymer scaffold. Therefore, in order to improve the efficiencies of cell seeding and chondrogenic differentiation, it is important to regulate the average diameter of cell aggregates appropriately.

In one embodiment of the present invention, the cell aggregates may be formed in a uniform size according to a hanging drop culture method. In another embodiment of the present invention, the cell aggregates of varying size may be formed according to a rotational culture method. First, the cells fully differentiated into chondrocytes through cultivation in a medium for chondrogenic differentiation are dispersed in the same medium in a proper cell concentration to prepare a cell suspension. When one drop of the cell suspension is applied onto the bottom of a culture dish followed by incubation, the cells proliferate in the drop hanging from the culture dish while simultaneously clustering together within several days, to thereby form cell aggregates of uniform size. Further, when the cell suspension prepared above is cultured in a rotating bioreactor while stirring at a constant rate, the cells proliferate while simultaneously clustering together within several days, to thereby form cell aggregates of varying size. The thus formed cell aggregates of varying size are passed through a sieve having a desired pore size, resulting in the separation and recovery of cell aggregates having an average diameter less than the pore size of the sieve.

In one embodiment of the present invention, the cell suspension used in the formation of cell aggregates may have a concentration in the range of 1×10⁴ to 1×10⁸ cells/ml. If the concentration of the cell suspension is too low, the cell aggregates may not be formed successfully and their average diameter may be too small. On the other hand, if the concentration of the cell suspension is too high, the average diameter of the cell aggregates may be too large, leading to a lowering of seeding and differentiation efficiencies. Therefore, in order to form cell aggregates having a desired size, i.e., in the range of 10 to 800 μm in diameter, it is important to maintain the cell suspension at a concentration of 1×10⁴ to 1×10⁸ cells/ml, more specifically, 1×10⁵ to 1×10⁶ cells/ml.

In the case of forming cell aggregates according to a rotational culture method, the average diameter of cell aggregates can be varied by regulating the stirring rate of the rotating bioreactor. Considering that conventional rotating bioreactors used in rotational culture are operated at a stirring rate of 20 rpm, the rotating bioreactor used in the present invention may be operated at a stirring rate in the range of 5 to 50 rpm so as to efficiently induce the formation of cell aggregates. The slower the stirring rate, the larger the average diameter of the cell aggregates is, while the faster the stirring rate, the smaller the average diameter of the cell aggregates is and cell aggregates of more uniform size in diameter are formed.

The cell aggregates of differentiated chondrocytes are mixed with hydrogels in a solution state, to thereby form a cell aggregate-hydrogel complex in which the cell aggregates are evenly dispersed in the hydrogel matrix. The cell aggregates and hydrogels may be mixed in a weight ratio in the range of 1:1 to 1:100. If the proportion of the hydrogel is not more than the above range, it is impossible to expect the role of hydrogels in establishing a three-dimensional environment physiologically similar to natural cartilage. On the other hand, if the proportion of the hydrogel exceeds the above range, the proportion of cell aggregates is proportionally decreased, and thereby, chondrogenesis-related ECM molecules are not sufficiently generated and secreted from the cell aggregates, which is unfavorable to cartilage regeneration.

Cartilage therapeutics using only cell aggregates has limited applicability in repairing a large area of cartilage damage due to the restricted number of chondrocytes included in the cell aggregates. Further, the use of cell aggregates only cannot provide the high mechanical strength necessary for retaining uniform morphology during the cartilage regeneration. Meanwhile, cartilage therapeutics using hydrogels and a single cell suspension is problematic in that, despite the use of hydrogels, it cannot induce efficient chondrogenic differentiation through intracellular interaction because of the use of single cells rather than cell aggregates. In order to overcome the above problems of conventional cartilage therapeutics, in one embodiment of the present invention, the differentiated chondrocytes are formed in cell aggregates having a proper size and the thus formed cell aggregates are then evenly dispersed in a hydrogel matrix, to thereby form a cell aggregate-hydrogel complex. As such, it is possible to establish a three-dimensional environment physiologically similar to natural cartilage and improve the efficiency of chondrogenic differentiation through intracellular interaction within the above environment. The physical properties and degradation rate of the hydrogel depending on its concentration can be varied according to the type of hydrogel used. In another embodiment of the present invention, the physical properties and degradation rate of the fibrin gel depending on its concentration are determined first, and then, the thus obtained results can be appropriately applied in each case where hydrogel is used.

In another embodiment of the present invention, cell aggregates having a diameter in the range of 10 to 800 μm that are formed by using the differentiated chondrocytes of 1×10³ to 1×10⁶ cells may be admixed with a hydrogel solution in a concentration of 0.05 to 10% at a weight ratio of 1:1 to 1:100. Here, if the hydrogel concentration is not more than 0.05%, the strength of the hydrogel would be too weak to act as a scaffold and easy to degrade before the chondrogenic differentiation and chondrogenesis of the cell aggregates are matured. On the other hand, if the hydrogel concentration exceeds 10%, chondrogenesis-related ECM molecules secreted from the cell aggregates are not successfully diffused and delivered, resulting in a slow progress in chondrogenesis. Suitable examples of hydrogels for the present invention may include, but are not limited to, fibrin, gelatin, collagen, hyaluronic acid, agarose, chitosan, polyphosphazine, polyacrylate, polyglactic acid, polyglycolic acid, pluronic acid, alginate, salts and the like. The above hydrogels may be used alone or as a mixture thereof.

The cell aggregate-hydrogel complex in which the cell aggregates of differentiated chondrocytes are evenly dispersed in the hydrogel matrix in a solution state is then seeded onto a polymer scaffold, followed by solidification of the hydrogel matrix into a gel state (gelation), to thereby obtain a cell aggregate-hydrogel-polymer scaffold complex. Such hydrogel solidification may be appropriately carried out by using the change in solidification temperature and pH, the addition of chemicals and so on depending on the type of hydrogel used. For example, in case of using fibrin as a hydrogel material, the mixture of the cell aggregates and fibrinogen is treated with thrombin and then immediately injected into a polymer scaffold. The thus injected fibrinogen in the solution state is spontaneously solidified into a gel state of fibrin after a few minutes, to thereby form a cell aggregate-fibrin-polymer scaffold complex in which the cell aggregate-fibrin complex is immobilized onto the surface of the polymer scaffold while filling up the pores thereof.

Cartilage is the portion to which various mechanical forces caused by body movement are applied. Since such mechanical forces play an important role in cartilage regeneration and chondrogenesis, cartilage therapeutics must have above a certain level of mechanical strength enough to sustain the pressure of the body load. The combined use of cell aggregates and hydrogels is favorable for the regeneration of functional cartilage having a similar structure to natural cartilage, but the mechanical strength is not sufficient to use in the treatment of damaged knee cartilage to which excessive mechanical load has been applied or in the treatment of a large area of joint defects. For improving cartilage regeneration efficiency while retaining high mechanical strength, flexibility, and uniform morphology during cartilage regeneration, in one embodiment of the present invention, the cell aggregate-hydrogel complex in a solution state is seeded onto the interconnective porous structure of a polymer scaffold, followed by immediately solidifying the hydrogel matrix into a gel state.

The polymer scaffold for the present invention may be a scaffold with a regular shape that is made of biodegradable and biocompatible polymers, where suitable examples of such polymers may include collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethyleneoxide)-poly(propyleneoxide)-poly(ethyleneoxide)copolymer, copolymers thereof and mixtures thereof, but are not limited thereto.

For the successful seeding of cell aggregate-hydrogel complex onto the polymer scaffold, the polymer scaffold should have an interconnective porous structure with a uniform pore size and exhibit high mechanical strength sufficient to sustain the internal body load.

Therefore, in one embodiment of the present invention, the polymer scaffold may have a pore size in the range of 10 to 1,000 μm. If the pore size is not more than 10 μm, the pore interconnectivity within the polymer scaffold is poor, while if the pore size exceeds 1,000 μm, the mechanical strength thereof is significantly lowered. Since the cell aggregates used in the present invention have an average diameter in the range of from 10 to 800 μm, when the seeding efficiency of the above cell aggregates, pore morphology, and the mechanical strength of a scaffold are considered, the polymer scaffold suitable for the present invention may have a pore size in the range of 10 μm to 800 μm, more specifically, 100 to 500 μm.

Further, the polymer scaffold suitable for the present invention may have a porosity of 40 to 97%. If the porosity of the polymer scaffold is not more than 40%, the pore interconnectivity therein is remarkably reduced, while if the porosity of the polymer scaffold exceeds 97%, the mechanical strength thereof is significantly lowered. When considering the pore morphology and mechanical strength of a polymer scaffold, the polymer scaffold used in the present invention may have a porosity in the range of 50 to 97%, more specifically 70 to 95%.

The polymer scaffold suitable for the present invention can be prepared by using the biodegradable and biocompatible polymer described above according to conventional methods well known in the art, such as, for example, casting/solvent extraction, gas foaming, phase separation, electrospinning, gel spinning, and the like.

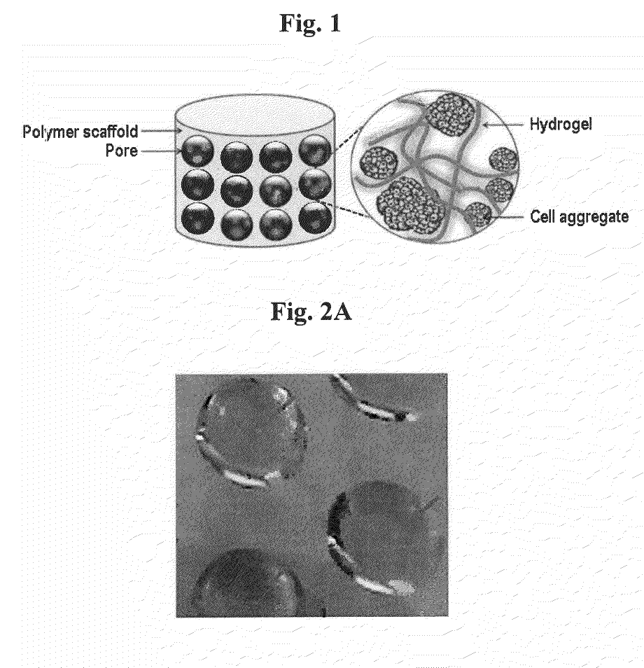

As shown in FIG. 1, the thus prepared cell aggregate-hydrogel-polymer scaffold complex according to the present invention has a structure where the cell aggregates of differentiated chondrocytes are evenly dispersed in the hydrogel matrix, to form a cell aggregate-hydrgel complex, and the cell aggregate-hydrgel complex is immobilized onto the surface of the polymer scaffold while simultaneously filling up the pores thereof.

The cell aggregate-hydrogel-polymer scaffold complex according to the present invention has the following advantages. First, it can efficiently induce chonrogenic differentiation due to the high degree of intracellular interaction resulting from the use of cell aggregates rather than single cells. In addition, since hydrogels creates three-dimensional environments physiologically similar to natural cartilage, a cell aggregate-hydrogel-polymer scaffold complex can further improve the efficiency of chondrogenic differentiation. Further, the use of a polymer scaffold enables the maintenance of high mechanical strength, flexibility and uniform morphology during the chondrogenic differentiation. Therefore, if the cell aggregate-hydrogel-polymer scaffold complex of the present invention is transplanted into a cartilage defect area of the patient, it can repair cartilage defects by regenerating cartilage tissue similar to natural cartilage, while retaining high mechanical strength, flexibility, and uniform morphology, and thus, can be effectively used as a cartilage therapeutic composition.

Another embodiment of the present invention includes a composition for cartilage regeneration including the cell aggregate-hydrogel-polymer scaffold complex of the present invention as an effective ingredient. The composition of the present invention can be effectively used in repairing cartilage defects caused by trauma, disease (such as osteoarthritis and osteochondrosis dissecans), excessive use of joints (e.g., sports injuries), other disruptions to the cartilage, or lifelong use of joints.

### EXAMPLES

Hereinafter, the embodiments of the present invention will be described in more detail with reference to the following examples. However, the following examples are only provided for purposes of illustration and are not to be construed as limiting the scope of the invention.

### Example 1

Bone marrows were collected from the tibia and fibula of 5-week old New Zealand white rabbits and subjected to density gradient centrifugation to separate bone marrow monocytes. The separated bone marrow monocytes were inoculated into a two-dimensional culture dish including 25 ml of DMEM supplemented with 10% serum and 1% penicillin/streptomycin in a concentration of 1×10⁷ cells/ml, followed by culturing in a 5% CO₂ incubator at 37° C. for 7 days. The medium was replaced with a fresh medium at intervals of 2 to 3 days. After the monolayer confluence reached 70% on the bottom of the culture dish, the cells were treated with 0.05% trypsin at 37° C. for 10 minutes to detach them from the culture dish. The thus obtained cells were inoculated into the same medium at a concentration of 1×10⁶ cells/ml and subjected to subculture under the same conditions to allow them to proliferate. Bone marrow-derived mesenchymal stem cells obtained after the third passage of subculture were treated with 0.05% trypsin at 37° C. for 10 minutes, followed by labeling with (continuous fluorescence depletion anisotropy (CFDA) fluorophore (Molecular Probe, USA) according to immunological methods. The CFDA-labeled bone marrow-derived mesenchymal stem cells were suspended in a medium for chondrogenic differentiation at a concentration of 5×10³ cells/20 μl (1 mM sodium pyruvate, 100 nM dexamethasone, 20 μg/ml proline, 37.5 μg/ml ascorbic 2-phosphate, 1% penicillin/streptomycin, 10 ng/ml TGF-β1, 1% FBS, 1× insulin-transferrin-selenium [ITS+]), to obtain a cell suspension. To prepare hanging drops, 20 μl/drop of the cell suspension was pipetted onto the bottom of a culture dish, which was then covered by a lid and tightly sealed. The sealed culture dish was kept in a 37° C. incubator for 7 days to induce the differentiation of bone marrow-derived mesenchymal stem cells into chondrocytes in the drop hanging from the culture dish, which were clustered together in the drop to form cell aggregates.

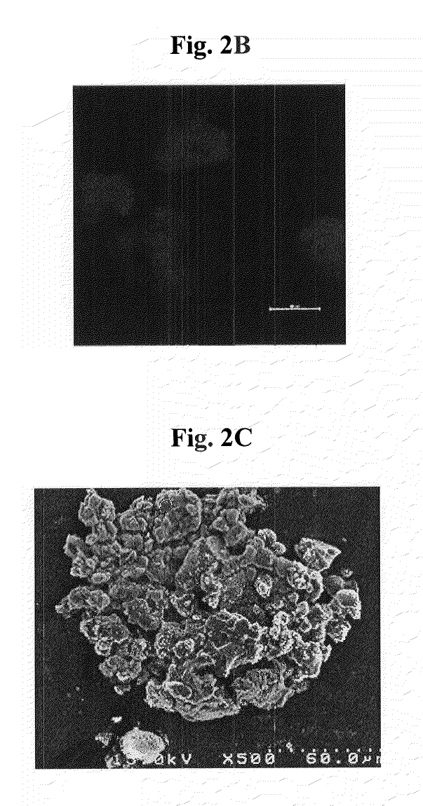

As shown in FIG. 2A, it was found that chondrocytes were differentiated in the form of cell aggregates from the bone marrow-derived mesenchymal stem cells. FIG. 2B is a fluorescent microscope photograph of the thus formed cell aggregates, illustrating that cell aggregates having a relatively uniform size of about 50 μm are observed as green fluorescence, which results from the cleavage of CFDA in the cytoplasm. The thus generated cell aggregates were dehydrated, dried, and observed with an electron microscope. As shown in FIG. 2C, it was found that a plurality of cells was clustered together and formed round-shaped aggregates.

The cell aggregates formed in the small drop hanging from the bottom of the culture dish were collected by washing the culture dish with an excess amount of the medium for chondrogenic differentiation. The collected medium including the cell aggregates was centrifuged gently to separate the cell aggregates therefrom, which were suspended in the same medium at a concentration of 200 cell aggregates/50 μl, so as to prepare a cell aggregate suspension. 50 μl of the cell aggregate suspension was mixed with 75 μl of a fibrinogen solution (Green Cross Corp, Korea) having a concentration of 5 mg/ml. After 75 μl of a thrombin solution at a concentration of 1 IU/ml was added thereto, the resulting mixture was immediately seeded onto a polymer scaffold. As a polymer scaffold, poly(lactide-co-carpolactone) (PLCL, 5:5) showing mechanical elasticity similar to natural cartilage and having a porosity of 85% and pore size in the range of 300 to 500 μm was used. After the seeding was completed, the polymer scaffold was kept in a 37° C. incubator so as to solidify the hydrogels seeded onto the polymer scaffold, to prepare a cell aggregate-hydrogel-polymer scaffold complex where the cell aggregate-hydrogel was immobilized onto the surface of the polymer scaffold while filling up the pores thereof.

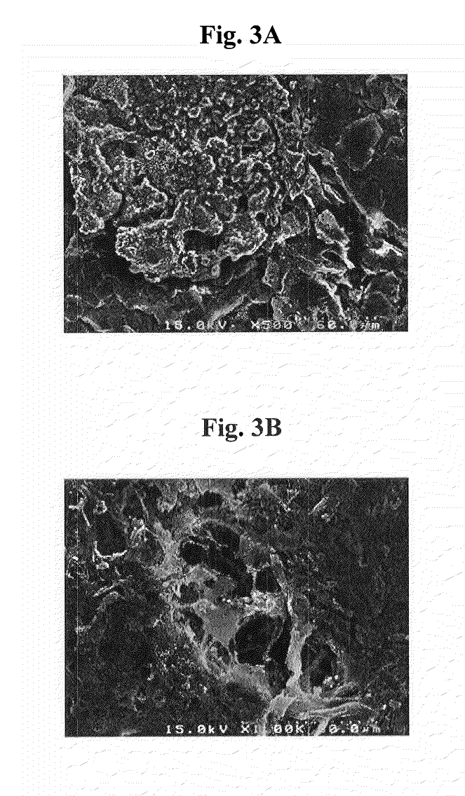

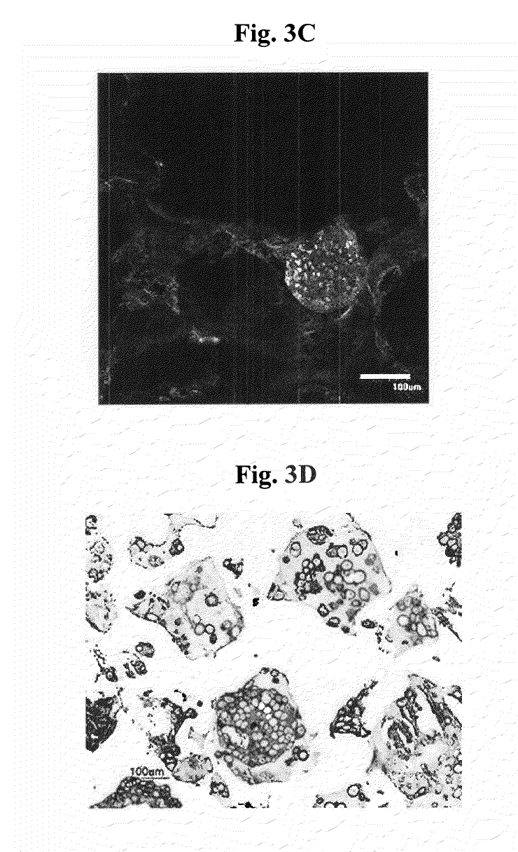

FIGS. 3A and 3B are electron microscope photographs of the thus prepared cell aggregate-hydrogel-polymer scaffold complex. As shown in FIG. 3B, at high magnification (×1000), it was found that the differentiated chondrocytes were clustered together in the form of cell aggregates and the cell aggregates were surrounded by fibrin fibers. As shown in FIG. 3C, an observation of the cell aggregate-hydrogel-polymer scaffold complex according to the present invention with a fluorescent microscope showed that it had a structure in which the cell aggregates and hydrogels were immobilized onto the surface of the polymer scaffold (nuclei—blue; actin fibers—red).

In order to examine the cartilage regeneration potential of the cell aggregate-hydrogel-polymer scaffold complex according to the present invention, the complex was subcutaneously transplanted into a nude mouse. Eight weeks after the transplantation, the complex was removed from the nude mouse and stained with Alcian blue so as to visualize the presence of cartilage-specific matrix molecules. As shown in FIG. 3D, it was found that lacuna structures typical to mature cartilage were evenly distributed at the surface of the complex. Further, the blue stained portions within the complex showed that cartilage-specific matrix molecules similar to natural cartilage have been successfully generated.

### Example 2

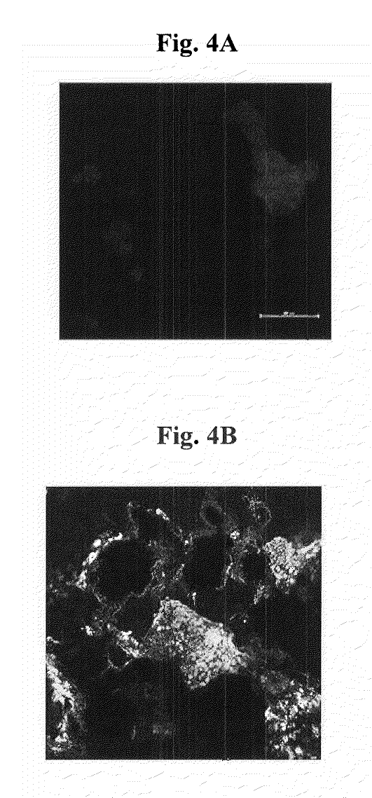

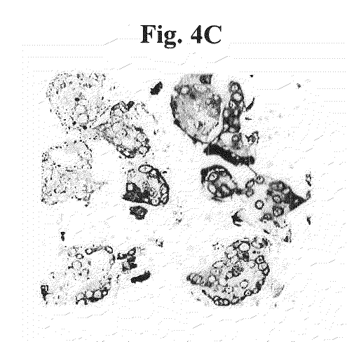

According to the same method as described in Example 1, bone marrow-derived mesenchymal stem cells were cultured, labeled with CFDA fluorophore, and suspended in a medium for chondrogenic differentiation at a concentration of 1×10⁵ cells/ml (1 mM sodium pyrubate, 100 nM dexamethasone, 20 μg/ml proline, 37.5 μg/ml ascorbic 2-phosphate, 1% penicillin/streptomycin, 10 ng/ml TGF-β1, 1% FBS, 1× insulin-transferrin-selenium [ITS+]), to obtain a cell suspension. 40 ml of the thus obtained cell suspension was placed in a rotating bioreactor and incubated in a 37° C. incubator for a week while stirring at a rate of 20 rpm, leading to the differentiation into chondrocytes and formation of cell aggregates. After the incubation was completed, cell aggregates of differentiated chondrocytes were formed in varying sizes, which were then passed through a sieve having a pore size of 700 μm to separate the cell aggregates having a diameter lower than 700 μm. The cell aggregates separated above were observed with a fluorescent microscope. As shown in FIG. 4A, it was found that the cell aggregates were formed in varying sizes ranging from 50 to 500 μm in diameter.

According to the same method as described in Example 1, after the cell aggregates were mixed with a fibrinogen solution having a concentration of 5 mg/ml, thrombin was added thereto, and the resulting mixture was immediately seeded onto a polymer scaffold. The thus prepared cell aggregate-hydrogel-polymer scaffold complex was observed with a fluorescent microscope. As shown in FIG. 4B, it was found that the cell aggregates of varying sizes were successfully introduced into the polymer scaffold (nuclei—blue; actin fibers—red).

According to the same method as described in Example 1, the cartilage regeneration potential of the cell aggregate-hydrogel-polymer scaffold complex prepared above was examined by transplanting the complex subcutaneously into a nude mouse, removing it from the nude mouse 8 weeks after the transplantation, and then, staining it with Alcian blue. As shown in FIG. 4C, it was found that in case of seeding the cell aggregates of varying sizes, lacuna structures typical to mature cartilage were evenly distributed at the surface of the complex. Further, the blue stained portions within the complex showed that cartilage-specific matrix molecules similar to natural cartilage were successfully generated.

While the present invention has been described and illustrated with respect to a number of embodiments of the invention, it will be apparent to those skilled in the art that variations and modifications are possible without deviating from the broad principles and teachings of the present invention which is defined by the claims appended hereto.

What is claimed:

1. A cell aggregate-hydrogel-polymer scaffold complex useful for cartilage regeneration comprising:
cell aggregates of differentiated chondrocytes;
a hydrogel matrix; and
a porous polymer scaffold,
wherein the cell aggregates are evenly dispersed in the hydrogel matrix and the resulting hydrogel matrix is immobilized onto the surface of the polymer scaffold while filling up the pores of the polymer scaffold.

2. The cell aggregate-hydrogel-polymer scaffold complex according to claim 1, wherein the cell aggregates are formed by differentiating cells having chondrogenic differentiation potential into chondrocytes and clustering them according to one of methods selected from the group consisting of hanging drop culture, pellet culture, micromass culture and rotational culture.

3. The cell aggregate-hydrogel-polymer scaffold complex according to claim 2, wherein the cells having chondrogenic differentiation potential are selected from the group consisting of mesenchymal stem cells and interstitial cells derived from any one of bone marrow, muscle, adipose, umbilical cord, amnion and amniotic fluid; precursor cells derived from said cells that can be differentiated into chondrocytes; chondrocytes differentiated from said cells; primary chondrocytes isolated from cartilage tissue; and mixtures thereof.

4. The cell aggregate-hydrogel-polymer scaffold complex according to claim 1, wherein the cell aggregates comprise 1×10³ to 1×10⁶ cells per aggregate and have an average diameter in the range of 10 to 800 μm.

5. The cell aggregate-hydrogel-polymer scaffold complex according to claim 1, wherein the hydrogel is selected from the group consisting of fibrin, gelatin, collagen, hyaluronic acid, agarose, chitosan, polyphosphazine, polyacrylate, polyglactic acid, polyglycolic acid, pluronic acid, alginate, salts thereof, and mixtures thereof.

6. The cell aggregate-hydrogel-polymer scaffold complex according to claim 1, wherein the polymer scaffold includes a polymer selected from the group consisting of collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethylene oxide)-poly(propylene oxide)-poly(ethylene oxide) copolymer, copolymers thereof, and mixtures thereof.

7. The cell aggregate-hydrogel-polymer scaffold complex according to claim 1, wherein the polymer scaffold has a pore size in the range of 10 to 1,000 μm and porosity in the range of 40 to 97%.

8. A method of preparing a cell aggregate-hydrogel-polymer scaffold complex comprising:
differentiating cells having chondrogenic differentiation potential into chondrocytes and clustering the chondrocytes to form cell aggregates;
dispersing the cell aggregates in a hydrogel matrix to prepare a cell aggregate-hydrogel complex; and
seeding the cell aggregate-hydrogel complex onto a porous polymer scaffold to immobilize said complex onto the surface of the polymer scaffold while filling up the pores of the polymer scaffold.

9. The method according to claim 8, wherein the cells having chondrogenic differentiation potential are selected from the group consisting of mesenchymal stem cells and interstitial cells derived from bone marrow, muscle, adipose, umbilical cord, amnion, or amniotic fluid, precursor cells derived from said cells that can be differentiated into chondrocytes, chondrocytes differentiated from said cells, primary chondrocytes isolated from cartilage tissue; and mixtures thereof.

10. The method according to claim 8, wherein the differentiating cells having chondrogenic differentiation potential into chondrocytes are carried out by a method selected from the group consisting of hanging drop culture, pellet culture, micromass culture, and rotational culture.

11. The method according to claim 8, wherein the cell aggregates contain 1×10³ to 1×10⁶ cells per aggregate and have a diameter in the range of 10 to 800 μm.

12. The method according to claim 8, wherein the dispersing the cell aggregates in a hydrogel matrix comprise mixing the cell aggregates and the hydrogel matrix in a weight ratio ranging from 1:1 to 1:100.

13. The method according to claim 8, wherein the hydrogel matrix is selected from the group consisting of fibrin, gelatin, collagen, hyaluronic acid, agarose, chitosan, polyphosphazine, polyacrylate, polyglactic acid, polyglycolic acid, pluronic acid, alginate, salts thereof, and mixtures thereof.

14. The method according to claim 8, wherein the hydrogel matrix is in a solution state having a concentration of 0.05 to 10%.

15. The method according to claim 8, the polymer scaffold comprises a polymer selected from the group consisting of collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethylene oxide)-poly(propylene oxide)-poly(ethylene oxide) copolymer, copolymers thereof, and mixtures thereof.

16. The method according to claim 8, wherein the polymer scaffold has a pore size in the range of 10 to 1,000 μm and a porosity in the range of 40 to 97%.

17. A composition for cartilage regeneration comprising the cell aggregate-hydrogel-polymer scaffold complex according to claim 1.
